(19) United States
(12) Patent Application Publication
(10) Pub. No.: US 2026/0363759 A1
(43) Pub. Date: Nov. 19, 2026

(54) INTEGRATED ELECTROSURGICAL SMOKE EVACUATION SYSTEM

(71) Applicant: Covidien LP, Mansfield, MA (US)

(72) Inventors: James D. ALLEN, IV, Broomfield, CO (US); Sarah R. WALDEN, Denver, CO (US); Daniel A. JOSEPH, Golden, CO (US); Jason M. MUCILLI, Lakewood, CO (US)

(21) Appl. No.: 19/135,491

(22) PCT Filed: Dec. 15, 2023
(86) PCT No.: PCT/IB2023/062771

§ 371 Date: Jun. 4, 2025

Related U.S. Application Data
(60) Provisional application No. 63/433,776, filed on Dec. 20, 2022.

(51) Int. Cl. A61B 18/14; A61B 18/00; A61B 18/12

(52) U.S. Cl. CPC A61B 18/1477; A61B 18/1206; A61B 2018/00702; A61B 2018/00922; A61B 2018/00946; A61B 2018/1273; A61B 2218/008

(57) ABSTRACT

An electrosurgical smoke evacuation system includes an electrosurgical pencil, a power unit, and a return electrode. The electrosurgical pencil is configured to deliver electrosurgical energy to tissue and evacuate smoke from a surgical site. The power unit is fluidly and electrically coupled to the electrosurgical pencil and includes an electrosurgical energy source, a suction generator configured to evacuate the smoke from the surgical site, and a filter configured to filter the smoke evacuated from the surgical site. The system also includes an actuator mechanism disposed on at least one of the electrosurgical pencil or the power unit. The actuator mechanism is configured to control at least one of a suction level of the suction generator or delivery of the electrosurgical energy to the electrosurgical pencil. The system also includes a return electrode configured for returning, to the electrosurgical energy source, the electrosurgical energy delivered to the tissue.

This Application claims priority from U.S. Provisional Patent Application 63/433,776, filed 20 Dec. 2022, the entire content of which is incorporated herein by reference.

### FIELD

The disclosure relates to electrosurgical devices. More specifically, the disclosure relates to an integrated electrosurgical smoke evacuation system.

### BACKGROUND

Monopolar electrosurgical(ES) pencils are used in surgery, typically for cutting tissue and/or for coagulating blood vessels. An ES pencil usually includes a handpiece into which electrodes of various shapes and sizes may be placed. The ES pencil is coupled to an ES generator, such as Medtronic's Valleylab™ FX8 or FT10 generator, which supplies the electrode with a high frequency, typically radio frequency (RF) alternating current. The ES generator may supply various waveforms suitable for achieving various surgical effects, such as cutting, coagulating, blending, spraying, fulgurating, and the like.

While using an ES pencil, surgical smoke is often generated. An effective way to evacuate surgical smoke from a surgical site is to use an ES pencil with an integrated smoke evacuation nozzle situated near the pencil's electrode in conjunction with a powered suction generator and an ultra-low penetration air (ULPA) filter. However, conventional smoke evacuation systems typically require various electrical and/or fluid connections from within the sterile field to outside the sterile field, which leads to electrical cords and tubing tangling and creating trip hazards. Moreover, smoke evacuation equipment may not always be available in an operating room. Even when smoke evacuation equipment is available, the level of suction may not be controllable from within the sterile field.

### SUMMARY

Provided in accordance with aspects of the present disclosure is an electrosurgical smoke evacuation system including an electrosurgical pencil, a power unit, and a return electrode. The electrosurgical pencil includes a handle housing, a nozzle defining a lumen for evacuating smoke from a surgical site, and an electrode configured to deliver electrosurgical energy to tissue. The power unit is fluidly and electrically coupled to the electrosurgical pencil and includes a housing. Disposed within the housing of the power unit is an electrosurgical energy source, a suction generator, a power source, and a filter. The electrosurgical energy source is configured to deliver electrosurgical energy to the electrode of the electrosurgical pencil. The suction generator is disposed within the housing and is configured to apply suction to the nozzle of the electrosurgical pencil for evacuating the smoke from the surgical site. The power source is disposed within the housing and is configured to power the electrosurgical energy source and the suction generator. The filter is disposed within the housing and is in fluid communication with the suction generator. The filter is configured to filter the smoke evacuated from the surgical site via the nozzle of the electrosurgical pencil. The return electrode is electrically coupled to the electrosurgical energy source of the power unit. The return electrode is configured to be adhered to a patient for returning, to the electrosurgical energy source, electrosurgical energy delivered to the tissue via the electrode of the electrosurgical pencil.

In an aspect of the present disclosure, the nozzle of the electrosurgical pencil is fluidly coupled to the suction generator of the power unit by a tube coupling the handle housing of the electrosurgical pencil to the suction generator.

In another aspect of the present disclosure, the electrode of the electrosurgical pencil is electrically coupled to the electrosurgical energy source by an electrosurgical supply line.

In another aspect of the present disclosure, the return electrode is electrically connected to the electrosurgical energy source by an electrosurgical return line.

In still another aspect of the present disclosure, the electrosurgical smoke evacuation system also includes an actuation mechanism disposed on the handle housing of the electrosurgical pencil, the actuation mechanism configured to control delivery of the electrosurgical energy to the electrode of the electrosurgical pencil.

In yet another aspect of the present disclosure, the electrosurgical smoke evacuation system also includes an actuation mechanism disposed on the handle housing of the electrosurgical pencil, the actuation mechanism configured to control a suction level of the suction generator.

In another aspect of the present disclosure, the electrosurgical smoke evacuation system also includes an actuation mechanism disposed on the housing of the power unit, the actuation mechanism configured to control delivery of the electrosurgical energy to the electrode of the electrosurgical pencil.

In another aspect of the present disclosure, the electrosurgical smoke evacuation system also includes an actuation mechanism disposed on the housing of the power unit, the actuation mechanism configured to control a suction level of the suction generator.

In still another aspect of the present disclosure, the housing of the power unit includes at least one exhaust vent in fluid communication with the suction generator.

In yet another aspect of the present disclosure, the power unit includes a liquid trap disposed within the housing and configured to collect liquid evacuated from the surgical site.

Another electrosurgical smoke evacuation system is provided in accordance with the present disclosure and includes an electrosurgical pencil, a power unit, an actuator mechanism, and a return electrode. The electrosurgical pencil is configured to deliver electrosurgical energy to tissue and evacuate smoke from a surgical site. The power unit is fluidly and electrically coupled to the electrosurgical pencil and includes a housing. The power unit also includes an electrosurgical energy source, a suction generator, and a filter. The electrosurgical energy source is disposed within the housing and is configured to deliver electrosurgical energy to the electrosurgical pencil. The suction generator is disposed within the housing and is configured to evacuate the smoke from the surgical site via the electrosurgical pencil. The filter is disposed within the housing and is configured to filter the smoke evacuated from the surgical site via the electrosurgical pencil. The actuator mechanism is disposed on at least one of the electrosurgical pencil or the power unit. The actuator mechanism is configured to control at least one of a suction level of the suction generator or delivery of the electrosurgical energy to the electrosurgical pencil. The return electrode is electrically coupled to the electrosurgical energy source of the power unit. The return electrode is configured to be adhered to a patient for returning, to the electrosurgical energy source, the electrosurgical energy delivered to the tissue via the electrosurgical pencil.

In an aspect of the present disclosure, the electrosurgical pencil is fluidly coupled to the suction generator of the power unit by a tube.

In another aspect of the present disclosure, the electrosurgical pencil is electrically coupled to the electrosurgical energy source by an electrosurgical supply line.

In still another aspect of the present disclosure, the housing of the power unit includes at least one exhaust vent in fluid communication with the suction generator.

In yet another aspect of the present disclosure, the electrosurgical pencil includes a nozzle defining a lumen in fluid communication with the suction generator for evacuating the smoke from the surgical site, and an electrode in electrical communication with the electrosurgical energy source and configured to deliver the electrosurgical energy to the tissue.

In another aspect of the present disclosure, the power unit includes a liquid trap disposed within the housing and configured to collect liquid evacuated from the surgical site.

In still yet another aspect of the present disclosure, the actuator mechanism is a slide switch configured to move longitudinally along a handle housing of the electrosurgical pencil.

Also provided in accordance with the present disclosure is an electrosurgical smoke evacuation kit. The electrosurgical smoke evacuation kit includes an electrosurgical pencil, a power unit, and a return electrode. The electrosurgical smoke evacuation kit also includes an electrosurgical supply line, a tube, and an electrosurgical return line. The electrosurgical pencil is configured to deliver electrosurgical energy to tissue and to evacuate smoke from a surgical site. The power unit is fluidly and electrically coupled to the electrosurgical pencil and includes a housing. The power unit also includes an electrosurgical energy source, a suction generator, a filter, and a power source. The electrosurgical energy source is disposed within the housing and is configured to deliver electrosurgical energy to the electrosurgical pencil. The suction generator is disposed within the housing and is configured to evacuate the smoke from the surgical site via the electrosurgical pencil. The filter is disposed within the housing and is configured to filter the smoke evacuated from the surgical site via the electrosurgical pencil. The power source is disposed within the housing and is configured to power the electrosurgical energy source and the suction generator. The electrosurgical supply line is configured to electrically couple the electrosurgical pencil to the electrosurgical energy source of the power unit. The tube is configured to fluidly couple the electrosurgical pencil to the suction generator of the power unit. The return electrode is electrically coupled to the electrosurgical energy source of the power unit. The return electrode is configured to be adhered to a patient for returning, to the electrosurgical energy source, the electrosurgical energy delivered to the tissue via the electrosurgical pencil. The electrosurgical return line is configured to electrically couple the return electrode to the electrosurgical energy source of the power unit.

In another aspect of the present disclosure, an actuator mechanism is disposed on at least one of the electrosurgical pencil or the power unit. The actuator mechanism is configured to control at least one of a suction level of the suction generator or delivery of the electrosurgical energy to the electrosurgical pencil.

In another aspect of the present disclosure, the power unit includes a liquid trap disposed within the housing and configured to collect liquid evacuated from the surgical site.

### BRIEF DESCRIPTION OF THE DRAWINGS

Aspects of the disclosure are described herein with reference to the accompanying drawings, wherein:

FIG. 1 is a perspective view of an integrated electrosurgical smoke evacuation system, in accordance with aspects of the disclosure;

FIG. 2 is a perspective view of the integrated electrosurgical smoke evacuation system of FIG. 1 illustrating a return electrode of the integrated electrosurgical smoke evacuation system coupled to a patient, in accordance with aspects of the disclosure;

FIG. 3 is a schematic view of a power unit of the integrated electrosurgical smoke evacuation system of FIG. 1, in accordance with aspects of the disclosure;

FIG. 4 is a perspective view of an electrosurgical(ES) pencil of the integrated electrosurgical smoke evacuation system of FIG. 1, in accordance with aspects of the disclosure;

FIG. 5 is an exploded perspective view of the ES pencil of FIG. 4, in accordance with aspects of the disclosure; and

FIG. 6 is a schematic illustration of an exemplary robotic surgical system configured for use with the integrated electrosurgical smoke evacuation system of FIG. 1, in accordance with aspects of the disclosure.

### DETAILED DESCRIPTION

Embodiments of the disclosure are now described in detail with reference to the drawings in which like reference numerals designate identical or corresponding elements in each of the drawings. The aspects may be combined in any manner consistent with the functionality of the apparatus and/or method disclosed herein. As used herein, the term “clinician” refers to a doctor, a surgeon, a nurse, or any other care provider and may include support personnel. Throughout this description, the term “proximal” will refer to the portion of the device or component thereof that is closer to the clinician and the term “distal” will refer to the portion of the device or component thereof that is farther from the clinician. As used herein, the term “exemplary” does not necessarily mean “preferred” and may simply refer to an example unless the context clearly indicates otherwise.

Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Additionally, in the drawings and in the description that follows, terms such as front, rear, upper, lower, top, bottom, and similar directional terms are used simply for convenience of description and are not intended to limit the disclosure. In the following description, well-known functions or constructions are not described in detail to avoid obscuring the disclosure in unnecessary detail.

The present disclosure relates to an “all-in-one” integrated electrosurgical smoke evacuation system including an ES pencil configured for evacuating surgical smoke during operation of the ES pencil to treat tissue. The ES pencil is electrically and fluidly coupled to a power unit, which integrates various components within a housing of the power unit to enable the ES pencil to treat tissue with monopolar electrosurgical energy and evacuate surgical smoke from a surgical site via suction applied to the ES pencil by the power unit. The power unit is light and portable and during a procedure can conveniently be placed on the patient, on the surgical bed next to the patient, or in proximity to the surgical bed within the sterile field. The power unit filters and/or traps the evacuated smoke and discharges filtered air into the surrounding environment. A return electrode is electrically coupled to the power unit and is configured to be adhered to the patient for returning the monopolar electrosurgical energy from the patient back to the power unit. The entire electrosurgical smoke evacuation system is configured to reside entirely within the sterile field during a procedure so that there is no need for electrical cords or tubing to extend from the sterile field into the surrounding area outside the sterile field. This arrangement minimizes trip hazards and tangling of electrical cords and/or tubes. The entire electrosurgical smoke evacuation system may be packaged in a single disposable package and set up of the system may simply include unpackaging the system within the sterile field, adhering the return electrode to the patient, and activating the power unit. Energy delivery and the application of suction may be controlled at the ES pencil and/or at the power unit within the sterile field, thereby eliminating the need for electrical connections to control interfaces outside of the sterile field, thereby further minimizing the number of electrical cords needed. To minimize expenses, the system may be returned to a manufacturer for reprocessing where certain components of the system may be disassembled, cleaned/disinfected, refurbished, re-assembled, and/or sterilized for reuse. Some of the system components may be discarded and/or recycled and replaced with replacement components.

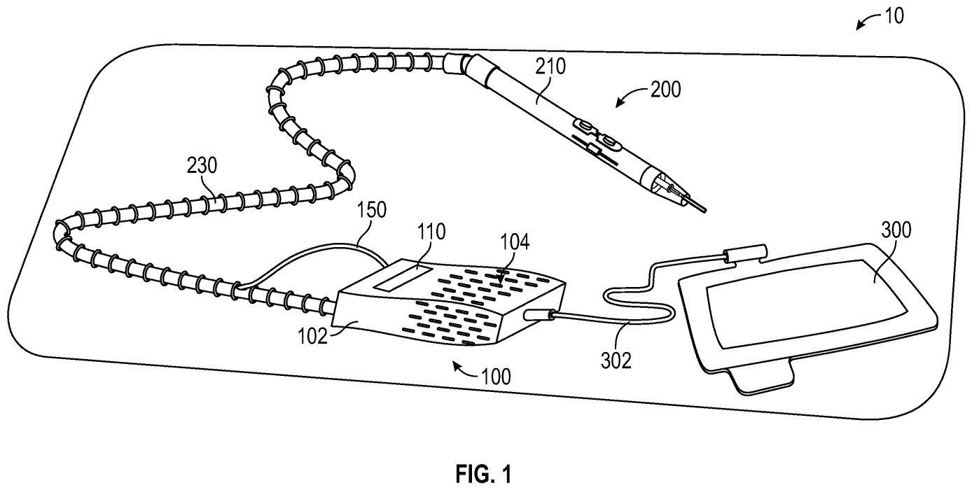

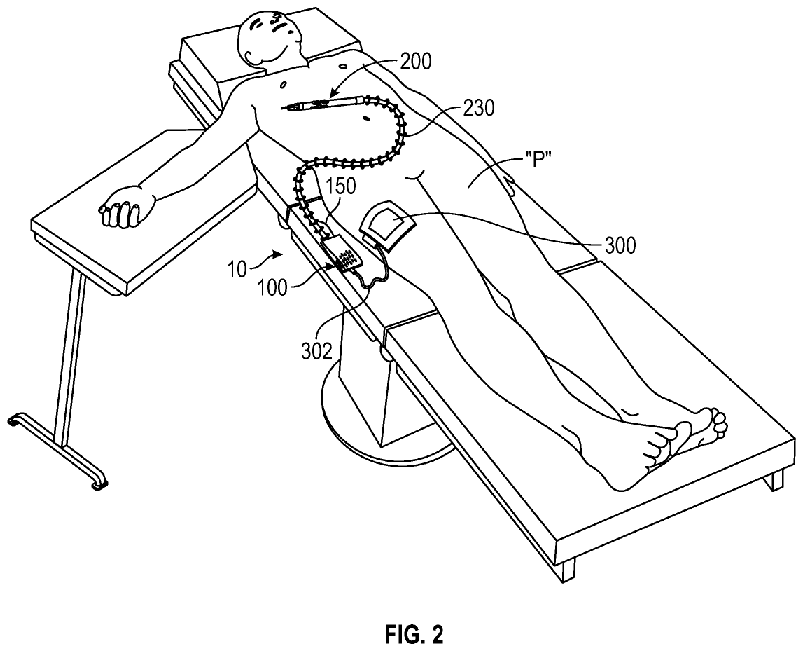

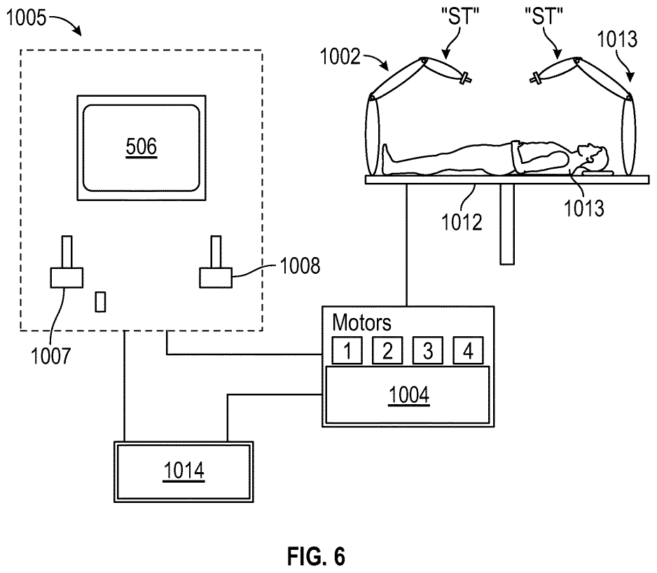

With reference to FIGS. 1 and 2, a surgical smoke evacuation system 10 in accordance with aspects of the present disclosure is shown. The surgical smoke evacuation system 10 generally includes a power unit 100 having a housing 102, an ES pencil 200 having a handle housing 210, and a return electrode 300 (e.g., a return pad). The ES pencil 200 is fluidly coupled to the power unit 100 by tubing 230 and electrically coupled to the power unit 100 by an electrosurgical supply line 150. The return electrode 300 is electrically coupled to the power unit 100 by an electrosurgical return line 302. During a procedure, the return electrode 300 is adhered to the skin of a patient “P” for returning, to the power unit 100, electrosurgical energy that is provided by the power unit 100 to the patient “P” via the ES pencil 200. The handle housing 210 of the ES pencil 200 may be configured as a handle configured to be gripped by a clinician, although non-handle configurations are also contemplated, e.g., for mounting the ES pencil 200 and/or attaching the ES pencil 200 to a surgical robot arm (see FIG. 6).

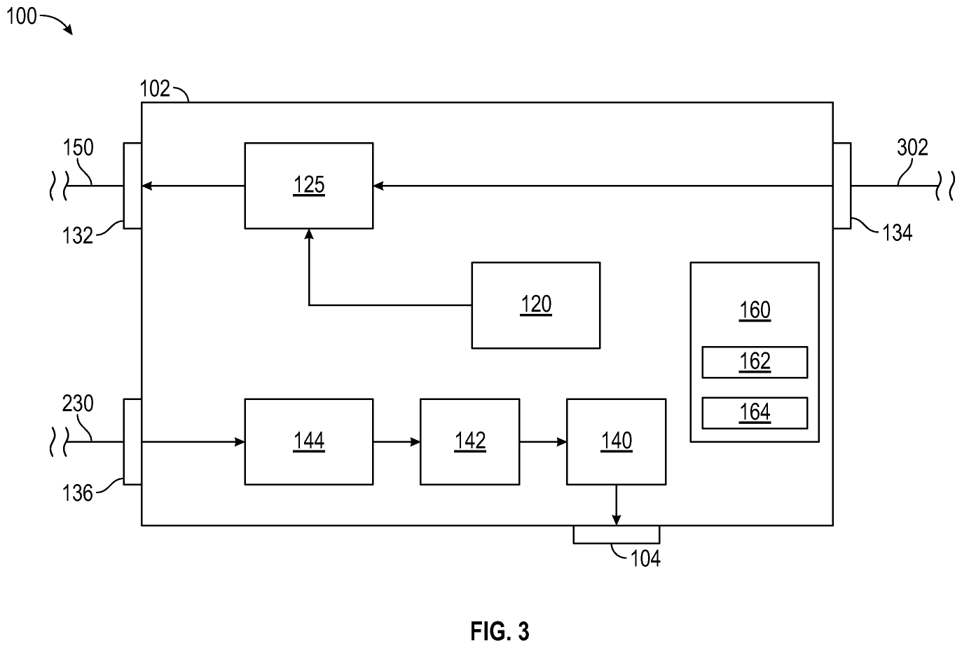

FIG. 3 shows a schematic block diagram of the power unit 100. Disposed within the housing 102 of the power unit 100 are a controller 160, a power source 120, an electrosurgical energy source 125, and a suction generator 144. In aspects of the present disclosure, the suction generator 144 may include a compressor pump. The power unit 100 is configured to output electrosurgical energy (e.g., radiofrequency, microwave, etc.) to the ES pencil 200 via the electrosurgical energy source 125 and to evacuate surgical smoke from a surgical site via suction applied to the ES pencil 200 by the suction generator 144. The controller 160, the electrosurgical energy source 125, and the suction generator 144 are powered by the power source 120. The power source 120 may include, for example, any one or more of a battery, a plurality of batteries, or a direct current high voltage power supply connected to an AC source (e.g., line voltage). Additionally or alternatively, the power unit 100 may be configured to receive power from a conventional AC wall outlet (not shown). In this scenario, the power unit 100 may include a suitable wire and plug (not shown) for electrically coupling one or more components of the power unit 100 to the AC wall outlet. The electrosurgical energy source 125 may be configured to convert the AC power provided from the AC wall outlet into electrosurgical energy suitable for use with the ES pencil 200.

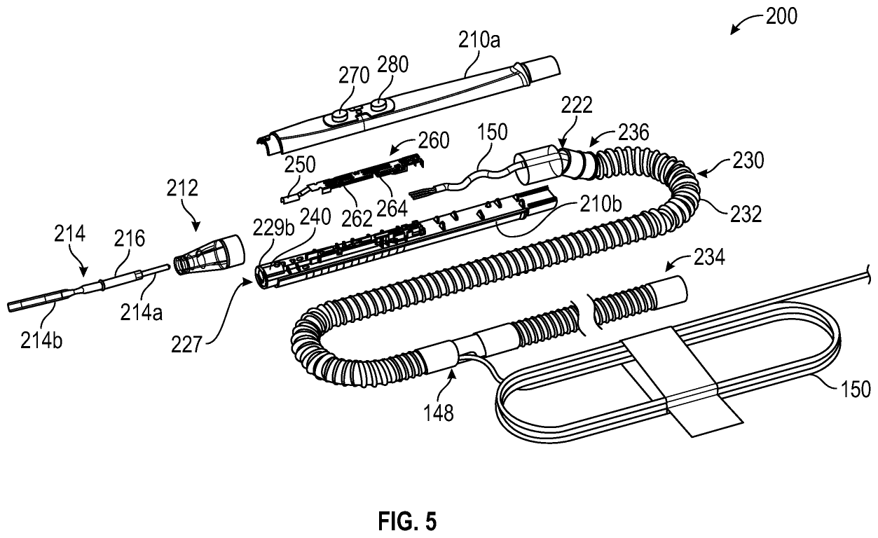

The suction generator 144 creates negative pressure having a vacuum force for removing surgical smoke from a surgical site during a procedure. The suction generator 144 may include a motor and one or more fans and/or pumps to create negative pressure for drawing surgical smoke from a surgical site into the power unit 100. The suction generator 144 is in fluid communication with an inlet port 136 disposed on the power unit 100. The inlet port 136 is configured for connection with a proximal end 234 of the tubing 230 (FIG. 5) to place the suction generator 144 in fluid communication with the ES pencil 200. In aspects of the present disclosure, the power unit 100 may be configured without the inlet port 136 and the proximal end 234 of the tubing 230 may be directly coupled to the suction generator 144. A filter 140 is disposed within the housing 102 in the power unit 100 in fluid communication with the suction generator 144 and the inlet port 136. The filter 140 allows evacuated air to pass through for discharge from the power unit 100 while trapping smoke particles within the filter 140. The filter 140 may also be configured to remove contaminants, debris, gaseous byproducts, and odors from the evacuated air. A liquid trap 142 is disposed within the housing 102 of the power unit 100 in fluid communication with the suction generator 144. To protect the filter 140 from getting wet, the liquid trap is configured to collect inadvertently suctioned liquid (e.g., blood, saline, etc.) prior to the liquid reaching the filter 140 such that the filter 140 is prevented from getting wet. Preventing the filter 140 from getting wet will increase the usable life of the filter 140. The suction generator 144 is in fluid communication with one or more exhaust vents 104 disposed through the housing 102 of the power unit 100 (see FIG. 1). The air that passes through the filter 140 is discharged from the power unit 100 through the one or more exhaust vents 104.

As shown in FIG. 3, the suction generator 144 is positioned within the housing 102 proximal to the filter 140 (e.g., the suction generator 144 is closer to the inlet port 136 and/or the tubing 230). By positioning the suction generator 144 proximal to the filter 140, pressure that is greater than atmospheric pressure is applied to the filter 140 during operation of the suction generator 144. The pressure through the filter 140 allows for the filter 140 to have a reduced surface area while still maintaining equivalent airflow through the filter 140. Positioning the suction generator 144 proximal to the filter 140 may also serve to increase the usable life of the filter 140. For example, as the filter 140 traps and accumulates particulates, the filter becomes clogged and the effective filter surface area is reduced. The suction generator 144 can compensate for clogging of the filter 140 by increasing the pressure applied to the filter 140 to maintain proper airflow through the filter 140. Although the liquid trap 142 is shown in FIG. 3 as positioned distal to the suction generator 144 (e.g., the liquid trap 142 is further from the inlet port 136 and/or the tubing 230), it should be understood that the liquid trap 142 may be disposed within and/or integral with the suction generator 144. In this scenario, the suction generator 144 may include a centrifugal pump that is configured to fling liquid into a housing of the suction generator 144 and collected in a reservoir of the liquid trap 142.

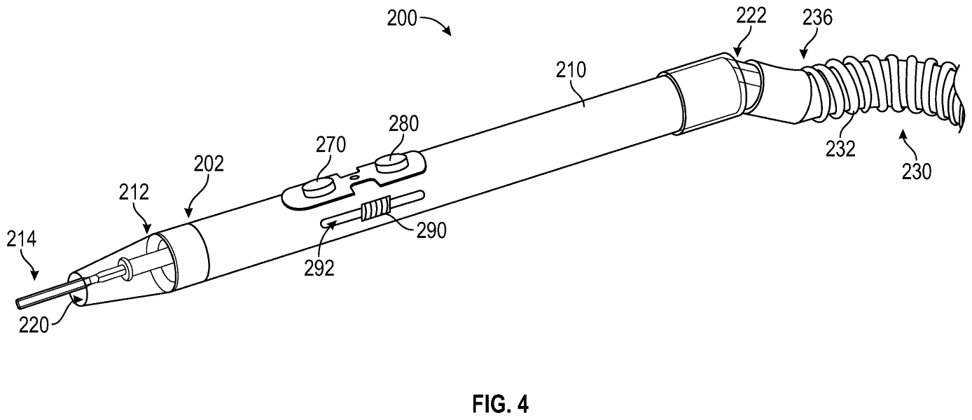

The electrosurgical energy source 125 provides electrosurgical energy to an electrode 214 (FIGS. 4 and 5) of the ES pencil 200 for treating (e.g., cutting, coagulating, ablating, etc.) tissue of the patient “P”. Electrosurgical energy is provided to the electrode 214 via the electrosurgical supply line 150. The electrosurgical supply line 150 is configured for connection to an active monopolar terminal 132 of the power unit 100. More specifically, the power source 120 provides DC power to the electrosurgical energy source 125, which converts the provided DC power into electrosurgical energy and delivers the electrosurgical energy to the active monopolar terminal 132 of the power unit 100. In aspects of the present disclosure, the power unit 100 may be configured without the active monopolar terminal 132 and the electrosurgical supply line 150 may be directly coupled to the electrosurgical energy source 125. As indicated above, AC power may be provided by an AC wall outlet. The electrosurgical energy is supplied to the patient “P” via the ES pencil 200 and returned to the electrosurgical energy source 125 through the return electrode 300 adhered to the patient “P” via an electrosurgical return line 302 that is connected to a return monopolar terminal 134 (FIG. 3) of the power unit 100. In aspects of the present disclosure, the power unit 100 may be configured without the return monopolar terminal 134 and the electrosurgical return line 302 may be directly coupled to the electrosurgical energy source 125. In aspects of the present disclosure, the surgical smoke evacuation system 10 may include a plurality of return electrodes configured to be disposed on the patient “P” to minimize the chances of tissue damage by maximizing the overall contact area with the patient “P”. In aspects of the present disclosure, the power unit 100 and the return electrode 300 may be configured for monitoring so-called “tissue-to-patient” contact to ensure that sufficient contact exists therebetween to minimize the chances of tissue damage.

The controller 160 has a processor 162 and a memory 164. Instructions may be executed by the processor 162, which may include one or more digital signal processors (DSPs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements. It is contemplated that the controller 160 may be located in the power unit 100, the ES pencil 200, and/or in a remote computer system.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Referring now to FIGS. 4 and 5, a connector 222 (e.g., swivel connector) at a distal end 236 of the tubing 230 couples the handle housing 210 to the tubing 230. The tubing 230 is in fluid communication with a lumen 227 extending through the handle housing 210 of the ES pencil 200. The lumen 227 of the handle housing 210 may be defined by the handle housing 210 itself or, in some aspects, may be a separate tube or luminal structure disposed within the handle housing 210 or coupled to an exterior of the handle housing 210. The tubing 230 may be corrugated by including a spiral spine 232 disposed on an outer surface of the tubing 230. The corrugated structure of the tubing 230 minimizes kinking and provides increased flexibility to the tubing 230. As shown in FIG. 5, the tubing 230 may also include an opening 148 at any point along its length for passage of the electrosurgical supply line 150 into a lumen defined within the tubing 230. A lumen defined through connector 222 is in fluid communication with the lumen defined through the tubing 230 and the lumen 227 of the handle housing 210. Thus, with the proximal end 232 of the tubing 230 connected to the inlet port 136 of the power unit 100, the lumen 227 of the handle housing 210 is placed in fluid communication with the suction generator 144 of the power unit 100 via the connector 222 and the tubing 230.

With reference to FIG. 5, the handle housing 210 includes a first housing portion 210a and a second housing portion 210b, which are secured to each other using any suitable method (e.g., ultrasonic welding) to secure and house internal components of the ES pencil 200. In aspects of the present disclosure, the handle housing 210 of the ES pencil 200 may be formed from a thermoplastic material.

The ES pencil 200 also includes a nozzle 212 coupled to a distal end portion of the handle housing 210. A lumen 220, e.g., a smoke lumen, is defined through the nozzle 212 for suctioning fluid (e.g., surgical smoke, debris, gaseous byproducts, etc.) from a surgical site and is in fluid communication with the lumen 227 defined by the handle housing 210. The lumen 227 defined by the handle housing 210 serves as a fluid intake to enable fluid (e.g., smoke) suctioned through the nozzle 212 to be evacuated through the handle housing 210 and the tubing 230 via operation of the suction generator 144. In aspects of the present disclosure, at least a portion of the nozzle 212 may be a transparent, substantially transparent, or translucent material configured to facilitate visual acuity in the surgical field. For example, at least a portion of the nozzle 212 may be formed from a clear polycarbonate resin. Other resin materials are contemplated such as, for example, polymethylmethacrylate or acrylic (PMMA), polymethylmethyacrylimide (PMMI), silicon-based resins, or the like. In aspects of the present disclosure, at least a portion of the nozzle 212 may be formed from a radiopaque material such as, for example, a thermoplastic polyurethane (TPU) material so that at least a portion of the nozzle 212 appears opaque under medical imaging modalities that use radiation such as X-rays for example.

The electrode 214 of the ES pencil 200 extends distally from a distal end portion 202 of the handle housing 210. The electrode 214 includes a distal portion 214b having a tissue treatment portion (e.g., a blade (as shown), a hook, a needle, etc.) and a proximal portion 214a disposed within the handle housing 210. The electrode 214 is removably received through a collet 216 that is, in turn, supported through a receptacle 240 formed within the handle housing 210 (FIG. 5) such that the collet 216 and the distal portion 214b of the electrode 214 extend distally from the distal end portion 202 and through the lumen 220 of the nozzle 212. The distal portion 214b of the electrode 214 includes a geometry that, in cooperation with an energy delivery algorithm stored in the memory 164 of the controller 160, serves to minimize electrical arcing and smoke plume at the surgical site. The residual smoke plume along with other fluids are captured by the nozzle 212 of the ES pencil 200 and drawn through the handle housing 210, the tubing 230, and into the power unit 100 by the suction generator 144. The evacuated smoke plume and other fluids are collected by the fluid trap 142 and/or filtered by the filter 140 before filtered air is discharged from the power unit 100 via the one or more exhaust vents 104.

In aspects, the electrode 214 is removable from the collet 216 such that the electrode 214 may be replaced by a new electrode and/or by an electrode having a different shape, size, and/or configuration depending on the needs of the clinician for a given procedure. The nozzle 212 may also be removable from the handle housing 210 such that the nozzle 212 may be replaced by a new nozzle and/or by a nozzle having a different shape, size, and/or configuration. For example, a nozzle may be replaced with a different size nozzle depending on the size of the electrode being used.

The proximal portion 214a of the electrode 214 extends proximally from the receptacle 240 and is received within an electrically conductive electrode clip 250 of an electrical unit 260 (FIG. 5) disposed within the handle housing 210. In aspects, the electrical unit 260 may be coupled to an interior surface (e.g., ribbing) of the handle housing 210 to secure and stabilize the proximal portion 214a of the electrode 214 within the handle housing 210. The electrical unit 260 is electromechanically coupled to the electrosurgical supply line 150, which interconnects the electrical unit 260 to the electrosurgical energy source 125 of the power unit 100. The electrical unit 260 includes a pair of switches 262, 264 (FIG. 5), which are aligned with a pair of push buttons 270, 280, respectively, extending from the first housing portion 210a, thereby allowing for activation of the switches 262, 264 when the corresponding button 270, 280 is pressed. Activation and/or deactivation of pushbutton switches 262, 264 serves to control delivery of electrosurgical energy from the power unit 100 to the electrode 214. For example, one of the buttons 270, 280 may serve to cause the electrosurgical energy source 125 of the power unit 100 to provide electrosurgical energy to the electrode 214 for cutting tissue and the other of the buttons 270, 280 may serve to cause the electrosurgical energy source 125 of the power unit 100 to provide electrosurgical energy to the electrode 214 for coagulating tissue.

In aspects of the disclosure, the buttons 270, 280 may be replaced by any suitable actuation mechanism, such as a rocker switch, a pressure sensitive transducer, or a slide switch configured to be actuated longitudinally (e.g., distally and proximally) along the handle housing 210 between multiple positions corresponding to energy output power levels of the electrosurgical energy source 125.

In aspects of the present disclosure, the ES pencil 200 may include an actuation mechanism 290 disposed within a longitudinal slot 292 (FIG. 5) on the handle housing 210. The actuation mechanism 290 is configured to be actuated longitudinally (e.g., distally and proximally) along the longitudinal slot 292 for enabling a clinician to control, within the sterile field and in real time during a procedure, a power level of electrosurgical energy being delivered from the electrosurgical energy source 125 of the power unit 100 to the electrode 214 of the ES pencil 200 and/or a suction level being applied by the suction generator 144 of the power unit 100 to the nozzle 212 of the ES pencil 200. In aspects, the actuation mechanism 290 may be a slide switch configured to be slid along the longitudinal slot 292 between multiple positions corresponding to power output levels of the electrosurgical energy source 125 and/or suction levels of the suction generator 144.

In aspects of the present disclosure, the power unit 100 may include an actuation mechanism 110 disposed on the housing 102 of the power unit 100. The actuation mechanism 110 is electrically coupled to any one or more of the internal components (e.g., the controller 160) of the power unit 100 to enable a clinician to control, within the sterile environment and in real time during a procedure, a power output level of electrosurgical energy being delivered from the electrosurgical energy source 125 of the power unit 100 to the electrode 214 of the ES pencil 200 and/or a suction level being applied by the suction generator 144 of the power unit 100 to the nozzle 212 of the ES pencil 200. In aspects of the present disclosure, the actuation mechanism 110 may be any suitable user interface and include, for example, a touch screen, one or more buttons, one or more switches (e.g., rocker switch), one or more pressure sensitive transducers, and/or one or more slide switches configured to be actuated longitudinally (e.g., distally and proximally) along the housing 102 of the power unit 100 between multiple positions corresponding to power output levels of the electrosurgical energy source 125 and/or suction levels of the suction generator 144 In aspects of the present disclosure, the surgical smoke evacuation system 10 may include either the actuation mechanism 110 of the power unit 100 or the actuation mechanism 290 of the ES pencil 200 for controlling the power output level and/or the suction level. In other aspects of the present disclosure, the surgical smoke evacuation system 10 may include both the actuation mechanism 110 of the power unit 100 and the actuation mechanism 290 of the ES pencil 200 for redundancy in controlling, in real time, the power output level and/or the suction level.

In aspects of the present disclosure, the power unit 100 may include or be coupled with a suitable mechanical interface configured to removable attach the power unit 100 to a structure within the sterile filed. For example, the power unit 100 may be attached to a surgical drape or a surgical bed to help maintain sterility of the power unit 100.

As is known in the art, surgical instruments can generally be categorized as reusable instruments (e.g., instruments that are cleaned and/or sterilized), disposable instruments (e.g., instruments that are entirely discarded after a single use), reposable instruments (e.g., instruments wherein portions are disposable and other portions are reusable after cleaning and/or sterilization), or reprocessable instruments. Typically, these reprocessable instruments are disposable instruments (or reposable instruments) that are collected after a surgical use and returned to a manufacturer where the instruments are disassembled, cleaned/disinfected, refurbished, re-assembled, sterilized, and sold as reprocessed instruments. In many cases, most of the original parts of an instrument are re-used to provide the necessary parts for reassembly of the same instrument. Parts that wear during use, are broken during disassembly, and/or are otherwise unable to be reprocessed as-is may be refurbished, modified, or discarded for recycling and replaced with replacement components. In aspects of the present disclosure, any one or more of the above-described components of the electrosurgical smoke evacuation system 10 may be sent to a manufacturer for reprocessing or discarded and/or recycled after a single-use. Costs associated with use of the disclosed electrosurgical smoke evacuation system 10 may be reduced by reprocessing as many components of the system as possible.

In aspects of the present disclosure, the above-described components of the surgical smoke evacuation system 10 may be provided in a common package as a kit. The kit includes the power unit 100, the ES pencil 200, the return electrode 300, the tubing 230, the electrosurgical supply line 150, and the electrosurgical return line 302 in a common package. In some aspects, the kit may include a plurality of electrodes of various sizes for replacing the electrode 214 of the ES pencil 200 and/or a plurality of nozzles of various sizes for replacing the nozzle 212 of the ES pencil 200. The kit may also include instructions for use (not shown). The common package of the kit may also include a thermoformed plastic tray (not shown) and/or other packaging materials within the purview of those skilled in the art.

Turning now to FIG. 6, a robotic surgical system 1000 configured for use in accordance with the present disclosure is shown. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a clinician, e.g., a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and a mounted device which may be, for example, a surgical tool “ST.” The surgical tools “ST” may include, for example, the ES pencil 200 of the present disclosure, thus providing any of the above-detailed functionality on a robotic surgical system 1000.

Robot arms 1002, 1003 may be driven by electric drives, e.g., motors, connected to control device 1004. The motors, for example, may be rotational drive motors configured to provide rotational inputs to accomplish a desired task or tasks. Control device 1004, e.g., a computer, may be configured to activate the motors, in particular by means of a computer program, in such a way that robot arms 1002, 1003, and, thus, their mounted surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Control device 1004, more specifically, may control one or more of the motors based on rotation, e.g., controlling to rotational position using a rotational position encoder (or Hall effect sensors or other suitable rotational position detectors) associated with the motor to determine a degree of rotation output from the motor and, thus, the degree of rotational input provided. Alternatively or additionally, control device 1004 may control one or more of the motors based on torque, current, or in any other suitable manner.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. An electrosurgical smoke evacuation system, comprising:
an electrosurgical pencil including:
a handle housing;
a nozzle defining a lumen for evacuating smoke from a surgical site; and
an electrode configured to deliver electrosurgical energy to tissue;
a power unit fluidly and electrically coupled to the electrosurgical pencil, the power unit having a housing and including:
an electrosurgical energy source disposed within the housing and configured to deliver electrosurgical energy to the electrode of the electrosurgical pencil;
a suction generator disposed within the housing and configured to apply suction to the nozzle of the electrosurgical pencil for evacuating the smoke from the surgical site;
a power source disposed within the housing and configured to power the electrosurgical energy source and the suction generator; and
a filter disposed within the housing and in fluid communication with the suction generator, the filter configured to filter the smoke evacuated from the surgical site via the nozzle of the electrosurgical pencil; and
a return electrode electrically coupled to the electrosurgical energy source of the power unit, the return electrode configured to be adhered to a patient for returning, to the electrosurgical energy source, electrosurgical energy delivered to the tissue via the electrode of the electrosurgical pencil.

2. The electrosurgical smoke evacuation system according to claim 1, wherein the nozzle of the electrosurgical pencil is fluidly coupled to the suction generator of the power unit by a tube coupling the handle housing of the electrosurgical pencil to the suction generator.

3. The electrosurgical smoke evacuation system according to claim 1, wherein the electrode of the electrosurgical pencil is electrically coupled to the electrosurgical energy source by an electrosurgical supply line.

4. The electrosurgical smoke evacuation system according to claim 1, wherein the return electrode is electrically connected to the electrosurgical energy source by an electrosurgical return line.

5. The electrosurgical smoke evacuation system according to claim 1, further comprising an actuation mechanism disposed on the handle housing of the electrosurgical pencil and configured to control delivery of the electrosurgical energy to the electrode of the electrosurgical pencil.

6. The electrosurgical smoke evacuation system according to claim 1, further comprising an actuation mechanism disposed on the handle housing of the electrosurgical pencil and configured to control a suction level of the suction generator.

7. The electrosurgical smoke evacuation system according to claim 1, further comprising an actuation mechanism disposed on the housing of the power unit and configured to control delivery of the electrosurgical energy to the electrode of the electrosurgical pencil.

8. The electrosurgical smoke evacuation system according to claim 1, further comprising an actuation mechanism disposed on the housing of the power unit and configured to control a suction level of the suction generator.

9. The electrosurgical smoke evacuation system according to claim 1, wherein the housing of the power unit includes at least one exhaust vent in fluid communication with the suction generator.

10. The electrosurgical smoke evacuation system according to claim 1, wherein the power unit includes a liquid trap disposed within the housing and configured to collect liquid evacuated from the surgical site.

11. An electrosurgical smoke evacuation system, comprising:
an electrosurgical pencil configured to deliver electrosurgical energy to tissue and evacuate smoke from a surgical site;
a power unit fluidly and electrically coupled to the electrosurgical pencil, the power unit having a housing and including:
an electrosurgical energy source disposed within the housing and configured to deliver electrosurgical energy to the electrosurgical pencil;
a suction generator disposed within the housing and configured to evacuate the smoke from the surgical site via the electrosurgical pencil; and
a filter disposed within the housing and configured to filter the smoke evacuated from the surgical site via the electrosurgical pencil;
an actuator mechanism disposed on at least one of the electrosurgical pencil or the power unit, the actuator mechanism configured to control at least one of a suction level of the suction generator or delivery of the electrosurgical energy to the electrosurgical pencil; and
a return electrode electrically coupled to the electrosurgical energy source of the power unit, the return electrode configured to be adhered to a patient for returning, to the electrosurgical energy source, the electrosurgical energy delivered to the tissue via the electrosurgical pencil.

12. The electrosurgical smoke evacuation system according to claim 11, wherein the electrosurgical pencil is fluidly coupled to the suction generator of the power unit by a tube.

13. The electrosurgical smoke evacuation system according to claim 11, wherein the electrosurgical pencil is electrically coupled to the electrosurgical energy source by an electrosurgical supply line.

14. The electrosurgical smoke evacuation system according to claim 11, wherein the housing of the power unit includes at least one exhaust vent in fluid communication with the suction generator.

15. The electrosurgical smoke evacuation system according to claim 11, wherein the electrosurgical pencil includes:
a nozzle defining a lumen in fluid communication with the suction generator for evacuating the smoke from the surgical site; and
an electrode in electrical communication with the electrosurgical energy source and configured to deliver the electrosurgical energy to the tissue.

16. The electrosurgical smoke evacuation system according to claim 11, wherein the power unit includes a liquid trap disposed within the housing and configured to collect liquid evacuated from the surgical site.

17. The electrosurgical smoke evacuation system according to claim 11, wherein the actuator mechanism is a slide switch configured to move longitudinally along a handle housing of the electrosurgical pencil.

18. An electrosurgical smoke evacuation kit, comprising:
an electrosurgical pencil configured to deliver electrosurgical energy to tissue and evacuate smoke from a surgical site;
a power unit fluidly and electrically coupled to the electrosurgical pencil, the power unit having a housing and including:
an electrosurgical energy source disposed within the housing and configured to deliver electrosurgical energy to the electrosurgical pencil;
a suction generator disposed within the housing and configured to evacuate the smoke from the surgical site via the electrosurgical pencil;
a filter disposed within the housing and configured to filter the smoke evacuated from the surgical site via the electrosurgical pencil; and
a power source disposed within the housing and configured to power the electrosurgical energy source and the suction generator;
an electrosurgical supply line configured to electrically couple the electrosurgical pencil to the electrosurgical energy source of the power unit;
a tube configured to fluidly couple the electrosurgical pencil to the suction generator of the power unit;
a return electrode electrically coupled to the electrosurgical energy source of the power unit, the return electrode configured to be adhered to a patient for returning, to the electrosurgical energy source, the electrosurgical energy delivered to the tissue via the electrosurgical pencil; and
an electrosurgical return line configured to electrically couple the return electrode to the electrosurgical energy source of the power unit.

19. The electrosurgical smoke evacuation kit according to claim 18, further comprising an actuator mechanism disposed on at least one of the electrosurgical pencil or the power unit, the actuator mechanism configured to control at least one of a suction level of the suction generator or delivery of the electrosurgical energy to the electrosurgical pencil.

20. The electrosurgical smoke evacuation kit according to claim 18, wherein the power unit includes a liquid trap disposed within the housing and configured to collect liquid evacuated from the surgical site.
